(19) United States
(12) Plant Patent Application Publication
(10) Pub. No.: US 2018/0139877 P1
(43) Pub. Date: May 17, 2018

(54) Apple tree named `Duke Fuji'

(50) ;

(71) Applicant: KIKU SRL GmbH, Girlan/Eppan (IT)

(72) Inventor: Danay Helmuth, Andrian (IT)

(73) Assignee: KIKU SRL GmbH, Girlan/Eppan (IT)

(21) Appl. No.: 15/732,102

(22) Filed: Sep. 18, 2017

(30) Foreign Application Priority Data
Nov. 11, 2016 (IT) 3624VV

(51) Int. Cl. A01H 5/08

(52) U.S. Cl. PLT/168

(57) ABSTRACT

‘Duke Fuji’ apple is sport mutation of ‘Brak’ Fuji notable for its distinctive fruit, which has an attractive solid red flush. ‘Duke Fuji’ colors early and evenly, allowing it to be harvested in a single picking.

Latin name: Malus domestica Borkh.

Variety denomination: ‘Duke Fuji’.

### BACKGROUND OF THE VARIETY

‘Duke Fuji’ is a new and distinct variety of apple tree (Malus domestica Borkh), which originated as a limb sport mutation of ‘Brak’ Fuji (U.S. Plant Pat. No. 15,261). ‘Duke Fuji’ was discovered by the inventor in a cultivated orchard at Andrian, Italy in 2009, and was selected for further observation because of the distinctive early and even coloring of its fruit. ‘Duke Fuji’ was first asexually propagated by chip budding in 2010 at Presina, Italy, and has since been observed to remain true to type over successive asexually propagated generations.

### BRIEF DESCRIPTION OF THE VARIETY

‘Duke Fuji’ was first selected for its distinctive fruit, which is fully and evenly colored well before harvest. Fruit of ‘Duke Fuji’ is also distinguished by its attractive deep red color and its readiness for harvest in a single pick.

‘Duke Fuji’ is distinguished from parent ‘Brak’ by its fruit, which exhibits a solid flush overcolor as compared to the striped overcolor of ‘Brak.’ Fruit of ‘Duke Fuji’ colors earlier than ‘Brak’ and can be picked in a single picking, as compared with two pickings required for ‘Brak.’

‘Duke Fuji’ is also distinguished from similar variety ‘Fubrax’ (U.S. Plant Pat. No. 18,761) by intense red solid flush overcolor of its fruit, as compared to the striped overcolor of ‘Fubrax.’ Young fruit of ‘Duke Fuji’ exhibits much greater anthocyanin coloration than young fruit of ‘Fubrax.’

‘GrofunFuji’ is distinguished from similar variety ‘Aztec’ by its fruit. ‘Aztec’ colors later and less evenly than ‘Duke Fuji,’ and exhibits a medium to dark red striping pattern over a yellow green ground as compared to the intense red solid flush overcolor of ‘Duke Fuji.’ ‘Aztec’ requires more than one picking, as compared to the single picking required for ‘Duke Fuji.’ The stem of ‘Aztec’ fruit is light brown in color as compared to the darker brown stem of ‘Duke Fuji.’

### BRIEF DESCRIPTION OF THE PHOTOGRAPHS

FIG. 1 shows blossoms, trunk and leaves of a ‘Duke Fuji’ tree;

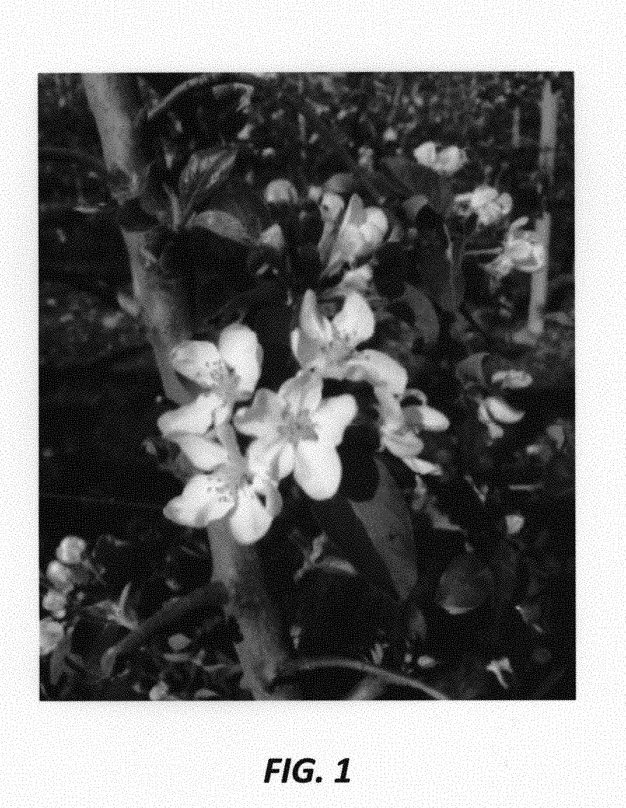

FIG. 2 shows a comparison of fruit color on ‘Duke Fuji’ and ‘Aztec’ trees;

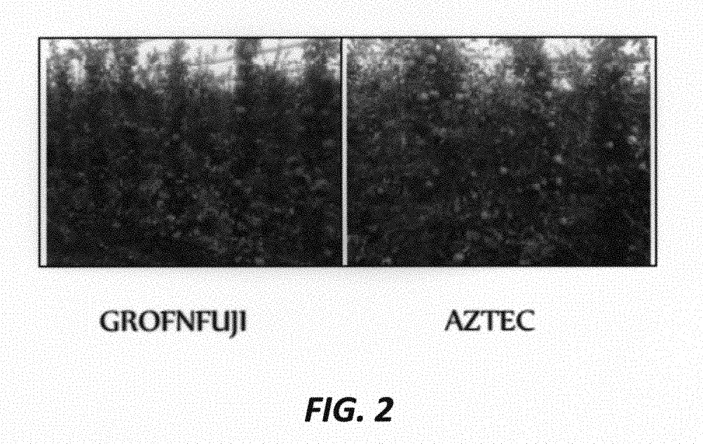

FIG. 3 shows a ‘Duke Fuji’ tree and fruit;

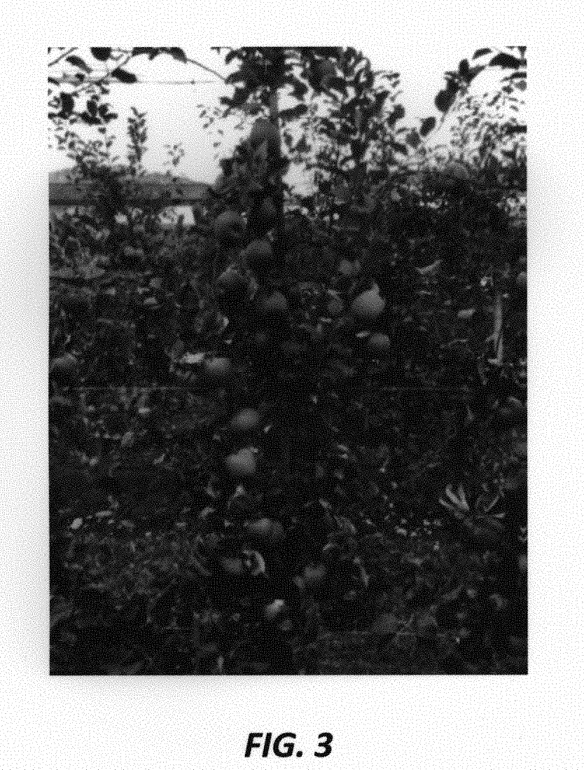

FIG. 4 shows fruit of ‘Duke Fuji’ (left), ‘Aztec’ (center) and ‘Fubrax’ (right);

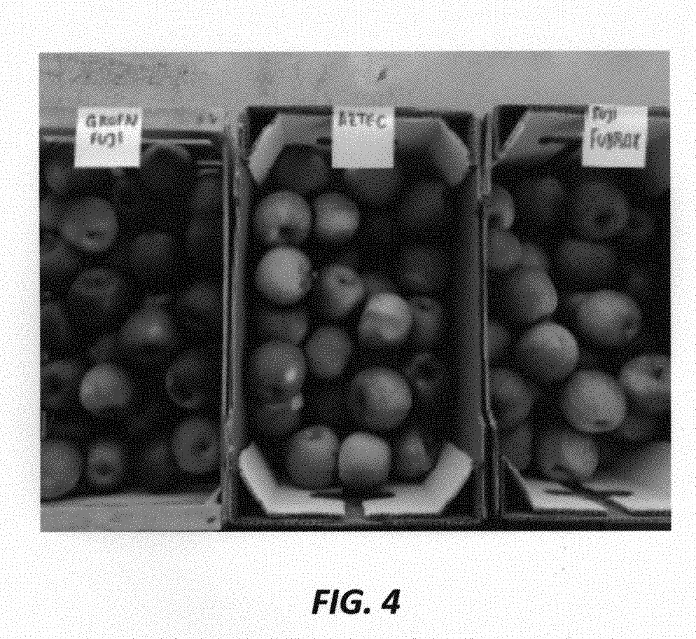

FIG. 5 shows young fruit of ‘Fubrax’ (left) and ‘Duke Fuji’ (right);

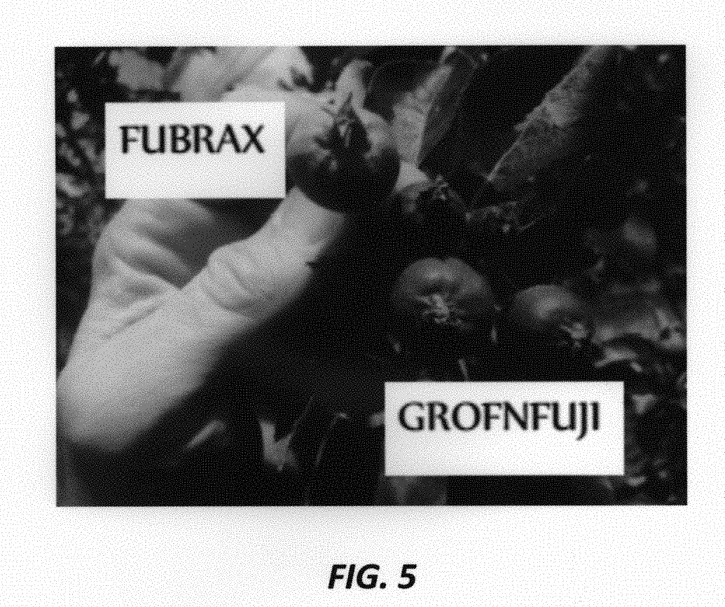

FIG. 6 shows fruit and leaves of ‘Duke Fuji’; and

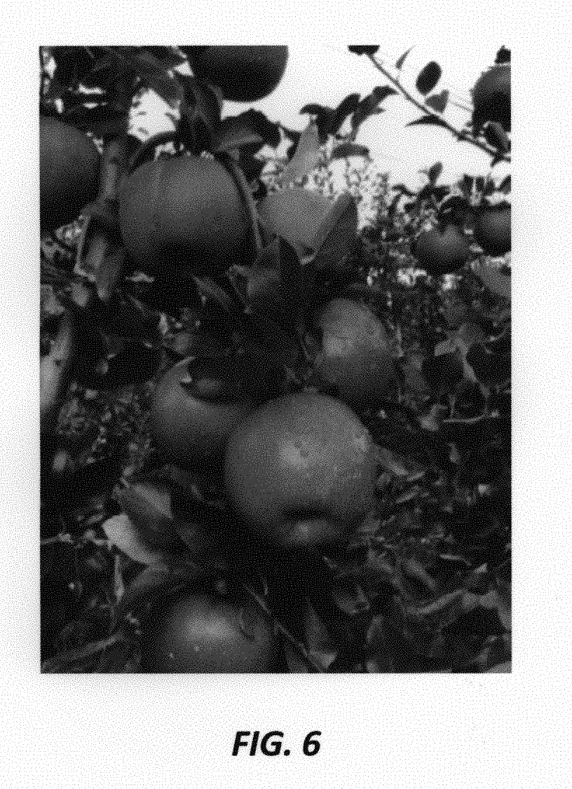

FIG. 7 shows leaves and whole and sectioned fruit of ‘Duke Fuji’.

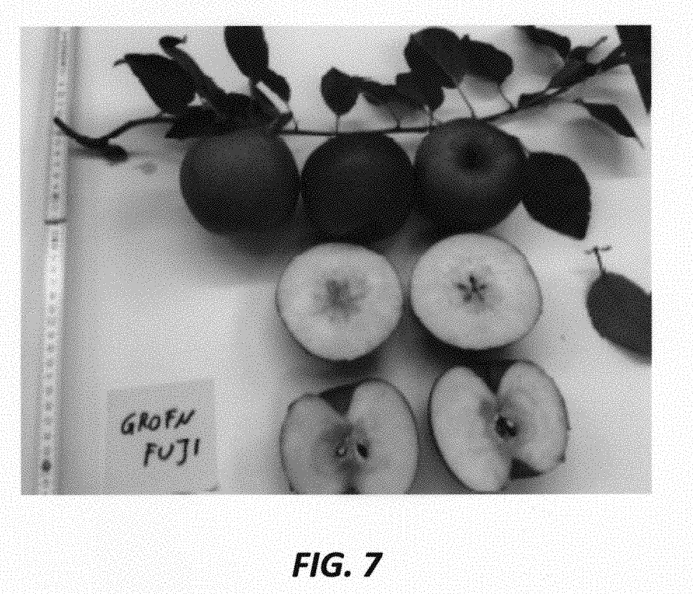

Actual colors will vary with lighting conditions; color characteristics of ‘Duke Fuji’ apple tree should therefore be determined with reference to the observations described herein below, rather than from these illustrations alone.

### DETAILED BOTANICAL DESCRIPTION OF THE VARIETY

The attached illustrations and the following detailed botanical description of ‘Duke Fuji’ apple tree are based on observations made and photos taken during the 2014 through 2017 growing seasons at Cornaiano, Italy of trees planted in 2012 on M9 T337 virus free rootstock. All colors are described according to The Royal Horticultural Society Colour Chart. The characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Tree:
   Vigor.—Strong, typical of ‘Fuji’ varieties.
   Type.—Ramified.
   Habit.—Drooping.
   Height.—1.7 m to 1.8 m (1 year old tree) 2.2 to 2.5 m (2year old tree).
   Trunk diameter at 30 cm above the graft.—12 mm to 16 mm (1 year old tree) 25 to 30 mm (2 year old tree).
   Bark texture.—Smooth.
   Bark color.—Greyed-green 197A.
   Type of bearing.—On spurs and long shoots.
Branch (fruiting branches located at around 1 m above the graft union):
   Length.—50 cm to 100 cm.
   Diameter.—1.5 cm +/−0.1 cm at 5 cm from the stem.
   Crotch angle.—35 to 45 degrees (1 and 2 year old tree) 55 to 60 degrees (3 year old tree).
   Bark color.—Brown N200B; Brown N200C on older trees.
   Lenticel length.—0.19 cm +/−0.03 cm.
   Lenticel color.—White 155A to 155B.
   Number of lenticels.—8 +/−4 per cm2.
One year old shoot:
   Length.—30 cm to 50 cm (1 year old tree) 50 to 60 cm (2 year old tree).
   Color.—Greyed-orange 177A.
   Pubescence.—Weak.
   Thickness.—0.8 cm +/−0.1 cm (medium).
   Internode length.—Medium.
   Number of lenticels.—Many, 8+/−6 per cm2.
Flower buds:
   Quantity per spur 6.
   Shape.—Oval.
   Length.—0.6 cm.
   Diameter.—0.4 cm.
   Color.—Greyed-purple N187A.
   Flowers.—Red 52A.
   Diameter of fully open flower 4 cm +/−0.5 cm.
   Relative position of petal margin.—Touching.
   Number per cluster.—5 plus king flower.
   Date of first bloom.—April 8 (typical of ‘Fuji’).
   Date of full bloom.—April 17.
   Pollination requirement.—Pollenizer such as ‘Gala’ required.
   Unopened flower.—Red 47A.
Petals (measured on king flower):
   Number per flower.—5.
   Shape.—Oval.
   Length.—2.4 cm +/−0.2 cm.
   Width.—1.4 cm +/−0.2 cm.
   Apex.—Round.
   Base.—Pointy.
   Margin.—Unruffled.
   Color of upper surface.—White N155D.
   Color of lower surface.—White N155D with slight traces of red 47A on edges.
Pistils:
   Size.—Vessels of pistil are erect, number from 10 to 12, length 5.5 mm.
   Color.—Green 138B.
Stigma:
   Position.—Above anthers (like ‘Golden Delicious’).
   Size.—0.08 cm +/−0.1 cm.
   Color.—Yellow 5A.
Style:
   Size.—1.0 cm +/−0.2 cm.
   Color.—Yellow-green 145C.
Ovary:
   Size.—0.5 cm +/−0.1 cm.
   Color.—Yellow-green 144A.
Anthers:
   Quantity.—10 +/−2.
   Size.—0.6 cm +/−0.1 cm.
   Presence of anther.—Yes.
   Color of anther.—Yellow-orange 17A.
Pedicel:
   Length.—3 cm +/−0.8 cm.
   Diameter.—0.16 cm +/−0.02 cm.
   Color.—Yellow-green 145A.
Sepals:
   Quantity.—5.
   Color.—Yellow-green 145A.
   Shape.—Pointed, triangular.
   Margin.—Smooth 0.75 cm +/−0.05 cm.
Leaves:
   Shape.—Elliptic.
   Length.—Up to about 10 cm.
   Width.—Up to about 6 cm.
   Length/width ratio.—2 +/−0.1 to 1.
   Blade margin.—Serrulate.
   Apex.—Acute.
   Base shape.—Aequilateral.
   Color of upper surface.—Green 137A to 137B for younger trees. Green 138A for older trees.
   Color of lower surface.—Green 137C to 138A for younger trees. Green 137C for older trees.
   Veins.—Color on lower surface red 47B.
   Attitude in relation to shoot.—Outward.
Petiole:
   Length.—2.6 cm to 3.6 cm.
   Diameter.—1.2 cm +/−0.1 cm.
   Color.—Green 138B.
Fruit:
   Quantity per cluster.—Up to 5.
   Diameter.—88 mm +/−8 mm.
   Weight.—270 g +/−25 g.
   Ratio of height to width.—1 to 1.
   General shape in profile.—Globose.
   Position of maximum diameter.—50% in profile.
   Ribbing.—Absent or weak.
   Crowning at calyx end.—Absent or weak.
   Depth of eye basin.—Small: 1 cm deep.
   Width of eye basin.—2.8 cm +/−0.2 cm.
   Length of sepal.—Medium, 0.45 cm.
   Bloom of skin.—Medium.
   Greasiness of skin.—Absent.
   Background color of skin.—Not visible.
   Amount of over color.—100%.
   Over color of skin.—Red 46A.
   Intensity of over color.—High.
   Pattern of over color.—Only solid flush.
   Amount of russet around stalk cavity.—Absent or small.
   Amount of russet on cheeks.—Absent.
   Area of russet around eye basin.—Absent.
   Length of stalk.—Short, 1.7 cm +/−0.3 cm.
   Thickness of stalk.—Medium, 1.8 cm +/−0.2 cm.
   Depth of stalk cavity.—Medium, 1.2 cm +/−0.1 cm.
   Width of stalk cavity.—Broad, 2.8 cm +/−0.1 cm.
   Firmness of flesh.—Medium, 6.5 kg per cm².
   Flesh texture.—Crunchy.
   Aroma.—Sweet.
   Juiciness.—High.
   Brix.—14 to 15.
   Flesh color.—Yellow 13D.
   Number of lenticels.—5 to 8 per cm².
   Size of lenticels.—Medium, up to 1.5 mm.
   Color of lenticels.—Greyed-yellow 160B.
   Aperture of locules.—Moderately open.
   Stem color.—Greyed-orange 177A.
Seeds:
   Quantity per fruit.—Up to 2 per chamber, up to 10 per fruit.
   Shape.—Pointed.
   Length.—0.8 cm +/−0.1 cm.
   Width 0.5 cm +/−0.1 cm.
   Color.—Greyed-orange 166C.
Harvest:
   Time for harvest.—Mid-October, similar to other mid-season ‘Fuji’-type varieties.
   Number of picks.—1.
   Amount of fruit produced per tree per harvest 25 kg +/−2 kg.
   Disease resistance/susceptibility.—None noted; same as other ‘Fuji’-type varieties.
   Market use.—Fresh.
   Storageability.—Up to 7 months in controlled atmosphere storage.

I claim:

1. A new and distinct apple tree, substantially as described and illustrated herein.
